(19) United States
(12) Patent Application Publication
(10) Pub. No.: US 2023/0404376 A1
(43) Pub. Date: Dec. 21, 2023

(54) TENSION CONTROL OF NONSYMMETRICAL FLEXIBLE DEVICES

(71) Applicant: INTUITIVE SURGICAL OPERATIONS, INC., Sunnyvale, CA (US)

(72) Inventors: Nicola Diolaiti, Menlo Park, CA (US); Mimi Trinh Fitterer, Belmont, CA (US); Samuel Raybin, Santa Clara, CA (US)

(21) Appl. No.: 18/252,435

(22) PCT Filed: Nov. 5, 2021
(86) PCT No.: PCT/US2021/058365

§ 371 Date: May 10, 2023

Related U.S. Application Data
(60) Provisional application No. 63/112,546, filed on Nov. 11, 2020.

(51) Int. Cl. A61B 1/005; A61B 1/018; A61B 1/00

(52) U.S. Cl. CPC A61B 1/0057; A61B 1/0055; A61B 1/018; A61B 1/00057; A61B 2090/0803

(57) ABSTRACT

Approaches to addressing asymmetric loads at the distal end of a flexible elongate device are disclosed herein. In some embodiments, symmetry is recovered by mechanically redesigning the flexible elongate device to counter the asymmetric load. In other embodiments, symmetry is recovered by altering the controls scheme for managing the control elements in a flexible elongate device. Generally, a flexible elongate device includes a plurality of control elements able to actuate the distal section. The amount of force applied to each control element can be changed so that the plurality of control elements is able to collectively apply a bending moment that counters the asymmetric load.

### CROSS-REFERENCE TO RELATED APPLICATION(S)

This patent document claims priority to and the benefit of U.S. Provisional Patent Application No. 63/112,546, filed Nov. 11, 2020, and incorporated herein by reference in its entirety.

### TECHNICAL FIELD

The present disclosure is directed to nonsymmetrical flexible devices having articulable portions controlled by tension-carrying control elements and associated systems and methods of use.

### BACKGROUND

Minimally invasive medical techniques are intended to reduce an amount of tissue that is damaged during medical procedures, thereby reducing patient recovery time, discomfort, and harmful side effects. Such minimally invasive techniques may be performed through natural orifices in a patient anatomy or through one or more surgical incisions. An operator may insert minimally invasive medical tools through these natural orifices or incisions to reach a target tissue location. Minimally invasive medical tools include instruments such as therapeutic, diagnostic, biopsy, and surgical instruments. One such minimally invasive technique is to use a flexible and/or steerable elongate device, such as a flexible catheter, that can be inserted into anatomic passageways and navigated toward a region of interest within the patient anatomy. Control of such an elongate device by an operator involves the management of several degrees of freedom including at least the management of insertion and retraction of the elongate device with respect to the patient anatomy, as well as steering of the device. To accomplish this, tension of the control elements extending along the length of the endoscope may be increased and/or decreased to cause articulation of the distal end.

### SUMMARY

Embodiments of the present technology are best summarized by the claims that follow the description.

Some embodiments pertain to a medical instrument system that comprises a medical instrument, a plurality of actuators, and a control system operably connected to the plurality of actuators. The medical instrument may comprise a flexible body having a distal end portion, a plurality of lumens along the flexible body including a lumen associated with an asymmetric load, and a plurality of control elements, each of which couples the distal end portion to a corresponding actuator of the plurality of actuators such that the plurality of actuators is operable to apply tension to the plurality of control elements to move the distal end portion. The control system may be configured to execute operations for determining a correction factor. The operations may comprise moving the distal end portion of the medical instrument to a plurality of distal tip positions by actuating the plurality of control elements and recording a plurality of measured tensions in the plurality of control elements to maintain each of the plurality of distal tip positions.

Other embodiments pertain methods for determining a correction factor for a flexible elongate device having an asymmetric load condition. The method may comprise moving a distal end portion of the flexible elongate device to a plurality of distal tip positions by actuating a plurality of control elements coupled to the distal end portion of the flexible elongate device and then recording a plurality of measured tensions in the plurality of control elements to maintain each of the plurality of the distal tip positions.

Further disclosed herein is a method of operating an elongate device to compensate for an asymmetric condition. The method may comprise determining a correction factor to compensate for the asymmetric condition and then applying a plurality of tensions to a plurality of control elements based on the correction factor. The plurality of control elements can be coupled to the distal end portion of the elongate device to enable actuation of the distal end portion. The correction factor may provide for maintaining the plurality of tensions at unequal preloads.

It is to be understood that the foregoing general description and following detailed description are explanatory in nature and intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure. The drawings should not be taken to limit the present disclosure to the embodiments that are depicted, as those embodiments were chosen for the purposes of explanation and understanding only.

FIGS. 1A-1C are simplified diagrams of a flexible elongate device configured in accordance with some embodiments of the present technology.

FIGS. 2A and 2B are simplified diagrams of models illustrating how a distal section of a flexible elongate device with a flexible body can be actuated using one or more control elements.

FIG. 3 is a simplified diagram of a model of a flexible elongate device with an asymmetrical load at the distal section.

FIG. 4 is a simplified diagram illustrating how symmetry can be recovered by repositioning control element(s) of a flexible elongate device that are arranged in the half plane opposite the load resulting in asymmetry.

FIG. 5A is a perspective view of a distal section of a flexible elongate device having an asymmetric design in accordance with an embodiment of the present technology.

FIG. 5B is a simplified cross-sectional diagram of the distal tip of the flexible elongate device of FIG. 5A.

FIG. 5C is a perspective view of a distal section of a flexible elongate device having an asymmetric design configured in accordance with another embodiment of the present technology.

FIG. 5D is a simplified side view of a distal tip portion of the flexible elongate device of FIG. 5C along with a side view of the distal tip portion of the flexible elongate device of FIGS. 5A and 5B.

FIG. 6A is a perspective view of a flexible elongate device with illumination fibers that have been rerouted near the distal tip in accordance with an embodiment of the present technology.

FIG. 6B is a partially schematic, cross-sectional view of the distal tip of the flexible elongate device of FIG. 6A.

FIG. 7 is a flow diagram of a process for determining the correction factor to compensate for the asymmetric condition of a flexible elongate device having a plurality of control elements in accordance with embodiments of the present technology.

FIG. 8 is a flow diagram of a process for operating a flexible elongate device with a plurality of control elements in an asymmetric condition in accordance with embodiments of the present technology.

FIG. 9 is a simplified diagram of a teleoperated medical system configured in accordance with various embodiments of the present technology.

FIG. 10A is a simplified diagram of a medical instrument system configured in accordance with embodiments of the present technology.

FIG. 10B is a simplified diagram of a medical instrument system configured in accordance with various embodiments of the present technology.

### DETAILED DESCRIPTION

The present disclosure is directed to nonsymmetrical flexible elongate devices having articulable portions controlled by tension-carrying control elements and associated systems and methods of use. The control elements can be used to control actuation of a distal section of a flexible elongate device with a lumen defined therethrough to provide a channel for a tool. Often, the flexible elongate device includes components that are provided in a number that cannot be evenly distributed around its perimeter while maintaining the lumen, so the distal section will experience an asymmetrical load. As explained in further detail herein, the control elements can be designed and/or controlled in such a manner to address such asymmetrical loads.

To facilitate delivery of a flexible elongate device to an intervention site (also referred to as a “target site”) as part of a procedure, the flexible elongate device may be sufficiently flexible to undergo substantial deformation yet axially rigid enough to avoid buckling. The flexible elongate device can include an articulable distal section having an axial support structure with a main lumen defined therethrough and grooves along its outer surface for accommodating control elements. For example, a plurality of control elements may be spaced evenly around the circumference of the axial support structure. To articulate the distal section of the flexible elongate device, actuation forces can be applied to the control elements.

Generally, the axial support structure will further include one or more grooves for accommodating other components, such as cable bundles, illumination fibers, and shape sensors. Incorporating these components, however, may cause the flexible elongate device to have asymmetric loading in the distal section. For instance, in some implementations a lumen containing the cable bundle for a camera and its cable bundle runs along a given side of the axial support structure, resulting in increased stiffness along that side of the axial support structure. In such a scenario, non-zero net torque will be experienced at the distal tip of the flexible elongate device when all of the control elements are experiencing the same actuation force due of the increased stiffness along the given side. This asymmetric load can be addressed in several different ways.

First, symmetry can be recovered by mechanically redesigning the flexible elongate device. For example, control element(s) that are arranged in the half plane opposite the lumen may be radially “pushed out” in order to increase their lever arms to counter the asymmetric load. Said another way, the control element(s) located in the half plane opposite the lumen may be relocated further from the longitudinal axis of the axial support structure. Relocating these control element(s) to be further from the longitudinal axis of the axial support structure will increase their lever arms, thereby countering the load associated with the lumen. Additionally or alternatively, control element(s) that are arranged in the same half plane as the lumen may be radially “pushed in” in order to decrease their lever arms to counter the asymmetric load. That is, the control element(s) located in the same half plane as the lumen may be relocated nearer to the longitudinal axis of the axial support structure. Note that an increase in load along a given side of a flexible elongate device will normally be caused by the stiffness of component(s) that are contained within the lumen(s) running along the given side. Accordingly, while a lumen may be said to be “responsible for” a load resulting in asymmetry, the load will be largely or entirely due to the stiffness of the component(s) contained in the lumen.

Second, symmetry can be recovered by altering the controls scheme for managing the series of control elements in a flexible elongate device. One option involves changing the actuation force applied to each control element so that when all control elements are at tension, the series of control elements will collectively apply a bending moment that counters the increased load. This approach may be referred to as “preloading” because when the flexible elongate device is commanded to enter a limp state (i.e., with net zero torque at the distal tip), the increased load will be compensated by the tension of some control elements being higher than other control elements. Another option involves measuring the articulation plane versus the commanded plane using a shape sensor located in the axial support structure of the flexible elongate device and then servo-ing the actuator(s) responsible for controlling the series of control elements into the commanded plane so that articulation of the control elements can be compensated accordingly.

The present application is directed to these approaches, each of which will be explained in further detail below. Note that, unless otherwise specified, aspects of these approaches can be applied in combination. Thus, flexible elongate devices that have been mechanically redesigned may implement the control schemes described herein to better address asymmetrical loads.

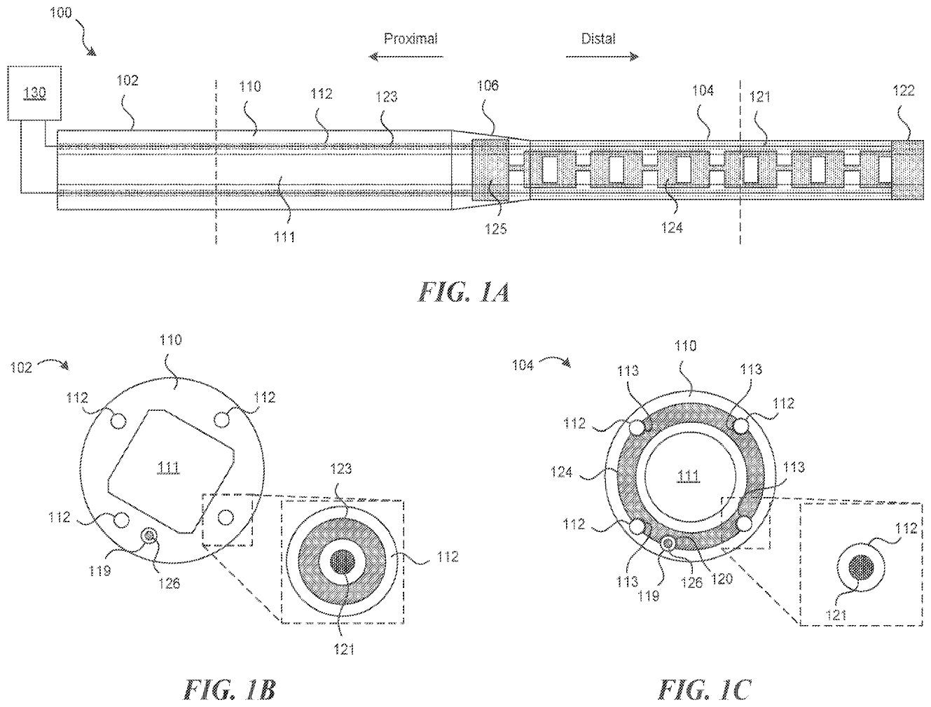

Specific details of several embodiments of the present technology are described with reference to FIGS. 1A-10B. Although embodiments may be described in the context of navigating the respiratory tract while performing a procedure, other applications are within the scope of the present technology. For example, unless otherwise specified, the devices, systems, and methods of the present technology may be used for navigation and treatment of anatomic tissues, via natural or surgically created connected passageways, in any of a variety of anatomic systems, including the lung, colon, stomach, intestines, kidneys and kidney calices, bladder, liver, gall bladder, pancreas, spleen, ureter, ovaries, uterus, brain, the circulatory system including the heart, vasculature, and/or the like.

It should be noted that embodiments in addition to those described herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein. Similarly, a person of ordinary skill in the art will understand that embodiments can be without some of the configurations, components, and/or procedures shown or described herein without deviating from the present technology.

This disclosure describes various instruments and portions of instruments in terms of three-dimensional space. The term “position” refers to the location of an object or a portion of an object in a three-dimensional space (e.g., the three degrees of translational freedom along Cartesian x-, y-, and z-coordinates). The term “orientation” refers to the rotational placement of an object or a portion of an object (e.g., the three degrees of rotational freedom—roll, pitch, and yaw). The term “pose” refers to the position of an object or a portion of an object in at least one degree of translational freedom and to the orientation of that object or portion of the object in at least one degree of rotational freedom (up to six total degrees of freedom). The term “shape” refers to a set of poses, positions, or orientations measured along an object.

The term “operator” shall be understood to include any person who may be performing or assisting a procedure, and thus is inclusive of physicians, surgeons, doctors, nurses, medical technicians, and other users of the present technology. The term “patient” should be considered to include human and/or non-human (e.g., animal) patients upon which a procedure is being performed.

### Introduction to Flexible Elongate Devices

FIGS. 1A-C are simplified diagrams of a flexible elongate device 100 configured in accordance with some embodiments of the present technology. This section provides an overview of certain features of the flexible elongate device 100, and the following section is directed to an overview of the control elements used to selectively actuate the flexible elongate device 100 during operation.

Referring first to FIG. 1A, the flexible elongate device 100 can include a proximal section 102, a distal section 104, and a transition section 106 therebetween. The flexible elongate device 100 comprises a flexible body 110 with a flexible wall having a thickness extending from an inner surface to an outer surface of the flexible body 110. A main lumen 111 (also referred to as a “working lumen”) can extend within the flexible body 110 through the proximal section 102, transition section 106, and distal section 104. The main lumen 111 can provide a delivery channel for a tool to be inserted through the flexible body 110. Examples of tools include medical instruments such as endoscopes, biopsy tools, imaging probes, ablation devices, chemical delivery mechanisms, and the like.

As shown in FIG. 1A, a plurality of control element lumens 112 may extend along the length of the flexible body 110. In this embodiment, the plurality of control element lumens 112 are arranged circumferentially around the main lumen 111 in the flexible wall. In some embodiments, a sensor lumen 119 also extends through the flexible wall of the flexible body 110. The sensor lumen 119 can extend from the proximal end 102 of the flexible elongate device 100, through the transition section 106, terminating at the distal end of the distal section 104. The flexible body 110 may include various other types of lumens for electrical wires, fibers, sensors, tools, and the like. Alternatively, the flexible body 110 may include an all-purpose lumen that can be used for a variety of purposes, including concurrently accommodating multiple tools, control elements, sensors, and the like.

Within each of the control element lumens 112, a conduit 123 (e.g., a coil pipe) may extend through the proximal section 102 of the flexible body 110, providing a channel through which a control element 121 extends. Examples of control elements 121 include pull wires, tendons, push rods, and the like. The conduits 123 may terminate at the transition section 106 proximal to the distal section 104. Thus, the control elements 121 may extend out of the conduits 123 at the transition section 106, entering the distal section 104 through control element lumens 112 and then attaching to a distal mount 122. The control elements 121 can be used to actuate the distal section 104 of the flexible elongate device 100 as will be further described below. In this example, four control elements 121 may be disposed within the control element lumens 112 and evenly spaced around the circumference of the flexible elongate device 100 as shown in FIGS. 1B-1C.

As further discussed below, the sensor lumen 119 and control element lumens 112 may be arranged around the main lumen 111 in various manners. In the example shown in FIG. 1B, a pair of lumens that includes sensor lumen 119 and a set of control element lumens 112 shown distributed around main lumen 111. In this example, the main lumen 111 is shown as a rounded square but it should be understood that the main lumen 111 may be of any shape. The control element lumens 112 are shown equally spaced around the rounded square but not centered along each side of the rounded square forming main lumen 111. Additionally, the sensor lumen 119 is not centered along the side of the main lumen 111. However, it should be understood that different numbers and/or arrangements of control element lumens 112 are possible. For example, the control element lumens 112 may be unevenly spaced around the circumference of the flexible elongate device and/or centered relative to the sides of a non-circular main lumen as further discussed below.

As further discussed with reference to FIG. 1C, the axial support structure 124 may have grooves in which components can be slidably inserted. In this example, the control element lumens 112 are located in corresponding grooves 113 while the sensor lumen 119 is located in corresponding groove 120. The distal mount 122 may be fixedly attached to the distal end of the flexible elongate device 100, and each control element 121 may be fixedly attached to the distal mount 122 but otherwise allowed to float within a corresponding groove in the axial support structure 124. Other grooves may be arranged around the circumference of the axial support structure 124 to accommodate cable bundles for cameras, illumination fibers, shape sensors, and the like.

Although the axial support structure 124 is illustrated as having a spine-like structure in FIG. 1A, other structures are possible. For example, the axial support structure 124 may be comprised of conduits similar to the conduits 123 in the proximal section 102. Whereas those conduits 123 are arranged concentrically around the control elements 121 to counteract the actuation forces applied to the control elements 121, the conduits of the axial support structure 124 may be offset from the control elements 121 (e.g., located at different positions around the circumference of the flexible body 110) to allow the axial support structure 124 to bend in response to actuation forces. Additionally or alternatively, the conduits of the axial support structure 124 may be more flexible (e g, smaller diameter and/or constructed with smaller gauge wire) than the conduits 123 in the proximal section 102. In some embodiments, the axial support structure 124 is formed as a single large coil that encloses the main lumen 111.

In some embodiments, one or more of the lumens 111, 112, 119 are keyed. That is, at least a portion of a lumen may have a non-circular cross-sectional shape that prevents or constrains the rotation of a tool (e.g., a medical instrument, sensor, fiber, electrical wire, or actuation element) with a matching non-circular cross-sectional shape when inserted through the lumen. Here, the proximal section 102 of the main lumen 111 is keyed as shown in FIG. 1B. In particular, the main lumen 111 has a rounded square cross-sectional shape that supports four keyed orientations.

In some embodiments, a localization sensor 126 extends through the sensor lumen 119. One example of a localization sensor 126 is a shape sensor (e.g., an optical fiber). Like the conduits 123, the localization sensor 126 may be constrained (e.g., fixedly attached and/or prevented from sliding axially) at one or both ends of the flexible elongate device 100. In some embodiments, the localization sensor 126 is fixedly attached to the distal mount 122 but freely floats within the sensor lumen 119. In some embodiments, a service loop is provided within the actuator 130 between a fixed localization attachment and a proximal end of the flexible elongate device 100 to accommodate the varying length of the sensor lumen 119 due to bending. Alternatively, the service loop can be provided between the actuator 130 and the distal end of the flexible elongate device 100 or within the flexible elongate device 100.

In some embodiments, the cross-sectional shape of the lumens 111, 112, 119 changes between the proximal section 102 and distal section 104. For example, the main lumen 111 may be keyed within the proximal section 102 and unkeyed within the distal section 104. As shown in FIG. 1C, the main lumen 111 can be unkeyed in the distal section 104, having a circular cross-sectional shape that does not constrain rotation of the tool inserted therein.

Similarly, the diameter of the lumens 111, 112, 119 may vary between the proximal section 102 and the distal section 104. Accordingly, the lumens 111, 112, 119 may be tapered in the transition section 106 to provide a gradual transition between different diameters and/or different cross-sectional shapes (e.g., a keyed lumen on the proximal side and an unkeyed lumen on the distal side).

In some embodiments, the flexible wall of the flexible body 110 varies between the proximal section 102 and distal section 104. For example, a required bending flexibility and/or compressive strength may vary along the length of the flexible elongate device 100 based on potential positioning within the patient anatomy. Thus, the flexible wall may include a plurality of layers that can vary within a proximal section of the flexible wall and within a distal section of the flexible wall.

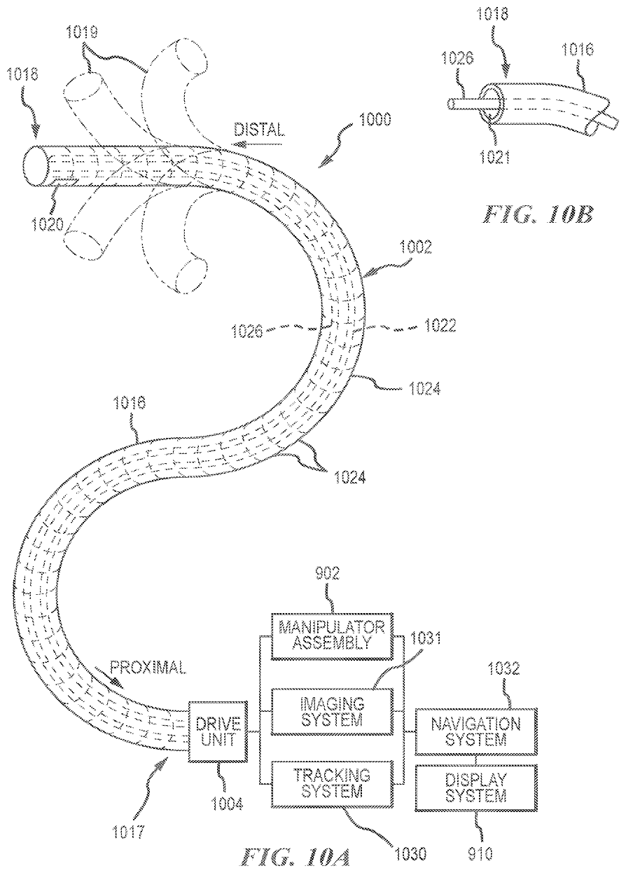

The flexible elongate device 100 may be navigated to a target site within patient anatomy by a medical instrument system. As the flexible elongate device 100 navigates the patient anatomy, the localization sensor 126 can generate data that is representative of the position, orientation, speed, velocity, pose, and/or shape of the distal section 104 of the flexible elongate device 100. As further discussed below with respect to FIGS. 10A-10B, this data can be sent to a navigation system that provides the operator with real-time position information that can be used to further navigate the patient anatomy and/or actuate the distal section 104.

As noted previously, the distal section 104 can be actuated by applying actuation forces to the control elements 121 (e.g., by pulling and/or pushing on the control elements 121 in an unequal manner). Applying actuation forces will cause the distal section 104 to bend in a direction defined by the net torque. The actuation forces may be applied manually, robotically, and the like. The actuation forces may be applied using one or more actuators 130 positioned at the proximal end of the flexible elongate device 100. Each of the plurality of actuators 130 is operable to control a corresponding control element of a plurality of control elements 121. In one example, the control elements 121 may be tendons or pull wires so that the actuators 130 provide for a change in tension within the tendons or pull wires.

The conduits 123 may transfer the actuation forces applied to the control elements 121 from the proximal end of the flexible elongate device 100 to the distal end of the proximal section 102 at the transition section 106. Consequently, even when unequal actuation forces are applied to the control elements 121, little actuation force may appear within the proximal section 102. In some embodiments, the conduits 123 are flexible to retain the flexibility of the proximal section 102.

Any bend along the length of the proximal section 102 of the flexible elongate device 100 will result in a change of length of the control element lumens 112. For example, with reference to FIG. 1A, if the flexible body 110 bends in a downward motion, the control element lumens 112 on the lower portion of the flexible body 110 will decrease in length while the control element lumens 112 on the upper portion of the flexible body 110 will increase in length. Thus, it may be necessary for the conduits 123 to axially slide within the control element lumens 112. In some embodiments, the conduits 123 are constrained (e.g., fixed and/or prevented from sliding proximally along a conduit longitudinal axis) at a proximal end of the flexible elongate device 100 within the actuator 130 and terminate at the transition section 106. In these embodiments, within the transition section 106, a stopper 125 may be coupled between the conduits 123 on the proximal side of the stopper 125 and an axial support structure 124 on the distal side of the stopper 125. The stopper 125 prevents the conduits 123 from shifting distally along the flexible elongate device 100. Alternatively, the conduits 123 may be fixed to the stopper 125. Further information on stoppers is provided in International Application No. PCT/US2018/043041, which is hereby incorporated by reference herein in its entirety.

Within the distal section 104, the axial support structure 124 can be configured to bend in response to actuation forces being applied to the control elements 121. Consequently, when unequal actuation forces are applied to the control elements 121, the distal section 104 will bend in the direction defined by the net torque. The axial support structure 124 supports the distal section 104 against axial loads generated by the actuation forces applied to the control elements 121. In particular, the axial support structure 124 may prevent or reduce distortion, compression, and/or collapse of the distal section 104 under axial loads.

### Modeling Tension of Control Elements

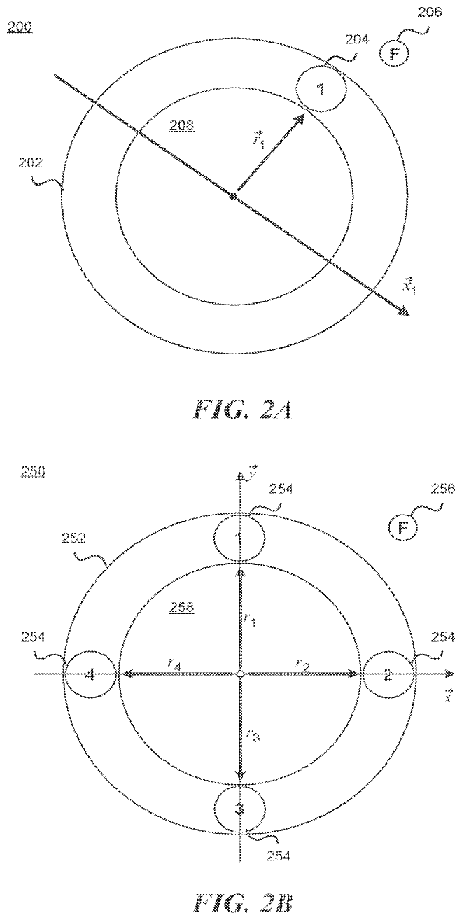

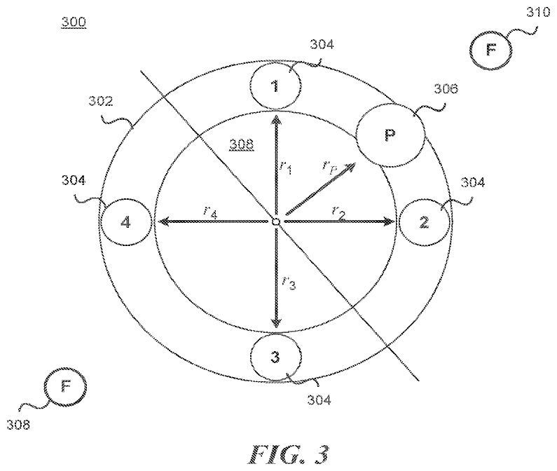

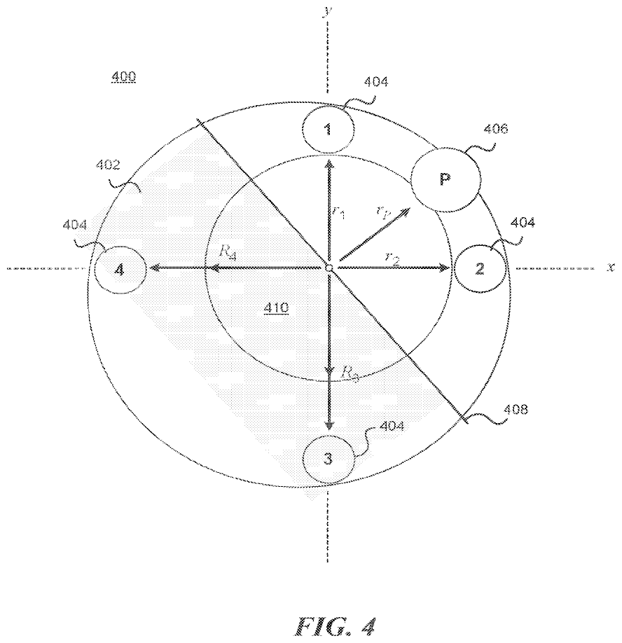

In one embodiment, to actuate the distal section in a desired direction, tension must be applied to the plurality of control elements in such a manner that the net torque acting on the distal section will cause movement in the desired direction. For purposes of illustration, several models that illustrate how net torque impacts movement are described below with reference to FIGS. 2A-2B and 3.

FIG. 2A, for example, is a simplified diagram of a model illustrating how a distal section of a flexible elongate device 200 with a flexible body 202 can be actuated using a single control element 204. When an actuation force is applied to the control element 204 (e.g., by pulling the proximal end of the control element 204 to increase tension), the distal section of the flexible body 202 will bend in the desired direction 206. When the actuation force is lessened (e.g., by reducing tension in the control element 204), the distal section of the flexible body 202 will recover to an initial configuration and, depending on the stiffness of the flexible body 202, may bend away from the desired direction 206. In FIG. 2A, r 1 represents the vector from the center of the main lumen 208 defined in the flexible body 202 to the radial location of the control element 204.

As discussed above, flexible elongate devices (such as the flexible elongate device 100 of FIG. 1A) can include a plurality of control elements that work in concert to actuate the distal section. FIG. 2B, for example, is a simplified diagram of a model illustrating how a distal section of a flexible elongate device 250 with a flexible body 252 can be actuated using multiple control elements 254. Here, the flexible elongate device 250 includes four control elements—Control Element 1, Control Element 2, Control Element 3, and Control Element 4. Other embodiments of the flexible elongate device 250 may include more than four control elements or fewer than four control elements.

Because the control elements 254 are arranged symmetrically about the main lumen 258 in the flexible body 252, all of the control elements 254 will have the same lever arm. That is, r₁, r₂, r₃, and r₄ will be the same. If the control elements 254 have the same properties (e.g., are comprised of the same material, have the same thickness, etc.), then all of the control elements 254 will have the same authority over control of the flexible elongate device 250. More specifically, when actuated in the same manner, all of the control elements 254 will be able to achieve the same maximum tension, the same bending moment (i.e., torque about \vec{x} and \vec{y}), and the same total displacement (i.e., in terms of bending in the positive and negative directions). Accordingly, when an operator provides input indicative of an instruction to actuate the distal section of the flexible elongate device 250 in a desired direction 256, to create an appropriate net torque, actuation forces will need to be applied to multiple control elements. Because of the symmetrical arrangement of the control elements 254, in order to bend the flexible elongate device 250 along \vec{y}, the appropriate net torque can be achieved by applying equal actuation forces to Control Element 3 and Control Element 1, where increasing tension in Control Element 3 and decreasing the same tension in Control Element 1 will cause bending in the direction of Control Element 3. Similarly, increasing tension in Control Element 1 and decreasing tension in Control Element 3 will cause bending in the direction of Control Element 1, and control of tensions in Control Elements 2 and 4 will cause bending along z.

This symmetrical control will be lost, however, if the structure of the flexible elongate device causes some control elements to be taxed more than others. FIG. 3, for example, is a simplified diagram of a model of a flexible elongate device 300 with an asymmetrical load at the distal section. In this embodiment, a lumen 306 extending along a given side of the flexible body 302 contains a component (e.g., a tool, a cable bundle for a camera, etc.) with a higher stiffness than the flexible body 302. The flexible body 302 will experience an increased load along the given side due to the increase in stiffness, which means that the control elements opposite the lumen 306 (e.g., Control Element 3 and Control Element 4) will have to apply larger actuation forces in some instances since those control elements need to overcome the increased load. For instance, Control Elements 3 and 4 would need to apply larger actuation forces to move the distal tip toward desired location 308 than Control Elements 1 and 2 would need to apply to move the distal tip toward designed location 310. To identify the control elements that are opposite the lumen 306, the vector (r p) from the center of the main lumen 308 to the location of the additional load (e.g. the additional component) is defined. Then, an axis orthogonal to the vector is identified.

Because of the increased load along the given side, there will be a non-zero net torque at the distal tip of the flexible body 302 when all of the control elements apply the same actuation force (e.g., are at the same tension). Accordingly, if the flexible elongate device 500 were commanded to enter a limp state (e.g., the tension in all control elements is reduced), the increased load of the lumen 506 will still produce a torque on the device and in some examples, the device may cause undesirable contact or force on the patient anatomy. The following discussion provides various techniques to address such asymmetrical loading conditions.

A. Mechanical Balancing of Control Elements

One approach to recovering control symmetry is mechanically balancing the construction of the flexible elongate device 400. FIG. 4, for example, is a simplified diagram of a flexible elongate device 400 configured in accordance with an embodiment of the present technology. More specifically, FIG. 4 illustrates how control symmetry can be maintained by positioning the control element(s) of the flexible elongate device 400 that are arranged in the half plane opposite the load resulting in control asymmetry. Much like the flexible elongate device 300 of FIG. 3, the flexible elongate device 400 includes a series of control elements 404 spaced evenly about the circumference of a flexible body 402 (e.g., each 90 degrees apart). Referring to FIG. 4, a load P is shown representing an asymmetrical element causing an unbalanced construction. The load P can be from a lumen 406 containing a component or plurality of components (e.g., a cable bundle for a camera, fiber, instrument, tool) running along a given side of the flexible body 402. The lumen 406 and/or the components can result in an additional stiffness causing the flexible body 402 to experience a load along the given side due to the increase in stiffness caused by inclusion of the component. The load P can represent a combination of asymmetrically located lumens, components held within the asymmetric lumens, components delivered within a main lumen 410 where the components are delivered offset from a central axis of the main lumen 410 or the components have an unbalanced construction, and structural asymmetry of the flexible body 402 (e.g., an asymmetric use of materials or non-uniform thickness of the flexible body wall).

To counter the load, control elements located radially opposite from the component causing an increased load may be positioned at a farther radial position from the central longitudinal axis of the flexible body 402 to increase respective lever arms in a manner that counters the increased load. For example, Control Element 3 and Control Element 4 have been positioned at a larger radial distance from the longitudinal axis of flexible body 402 than Control Element 1 and Control Element 2, so that lever arms (R₃ and R₄) collectively counter the lever arms of Control Element 1 (r₁), Control Element 2 (r₂), and the load (rₚ). Additionally or alternatively, the control elements (e.g., Control Element 1 and Control Element 2) arranged in the same half plane as the component causing an increased load (e.g., lumen 406), may be located closer to the longitudinal axis of the flexible body 402. To provide for the uneven radial spacing of the lumens for the control elements 404, the thickness of the flexible body 402 may be asymmetric. Accordingly, the flexible body 402 may vary in thickness to accommodate all of the control elements 404. The control elements 404 should be radially positioned in such a manner that the main lumen 410 remains usable for delivery of tools and instruments.

The desired radial locations of the control elements 404 can be determined by modeling stiffness of the load as a function of bending angle and then assuming a known circumferential position of the lumen 406 with respect to the x- and y-axis. Modeling may be particularly useful when the loads associated with more than one component need to be accounted for. As an example, a flexible elongate device may include a cable bundle for a camera and a single illumination fiber along opposing sides of the flexible body. In such embodiments, modeling stiffness may be helpful in establishing how the net torque is affected by the load of the cable bundle being partially countered by the load of the illumination fiber.

Additionally or alternatively, the control elements 404 may have different properties which can balance the control asymmetry. For example, a first subset of the control elements (e.g., Control Elements 1 and 2) may be tensioned wires having a first diameter, while a second subset of the control elements (e.g., Control Elements 3 and 4) may be tensioned wires having a second diameter different than the first diameter. The first and second diameters may be selected to mitigate the impact of asymmetry on bending in the distal section of the flexible elongate device 400. As another example, a first subset of the control elements (e.g., Control Elements 1 and 2) may be comprised of a first material, while a second subset of the control elements (e.g., Control Elements 3 and 4) may be comprised of a second material different than the first material. The first and second materials may be selected to mitigate the impact of asymmetry on bending in the distal section of the flexible elongate device 400. In some embodiments, depending on the construction of the flexible elongate device 400 and the tools and instruments to be delivered within the main lumen 410 of the flexible elongate device 400, each of the control elements 404 may be constructed from different materials and/or of different diameters. In some embodiments, one or more of the control elements 404 includes a service loop at the proximal end (e.g., within the flexible body 402). When an instrument delivered through the main lumen 410 is bent, the service loop(s) may facilitate extension of the corresponding control element(s) along an outer bend.

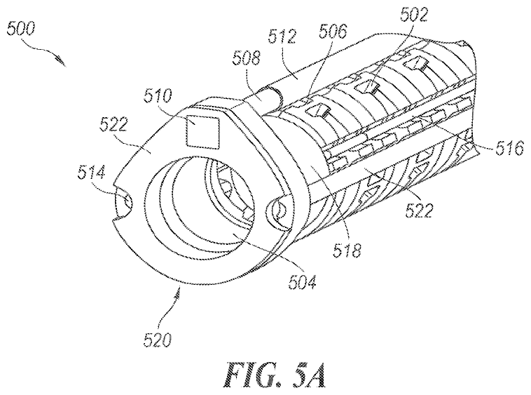

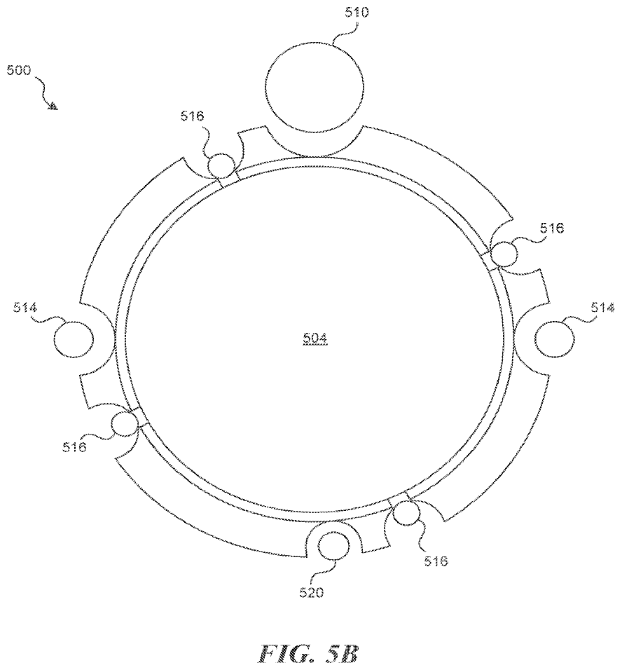

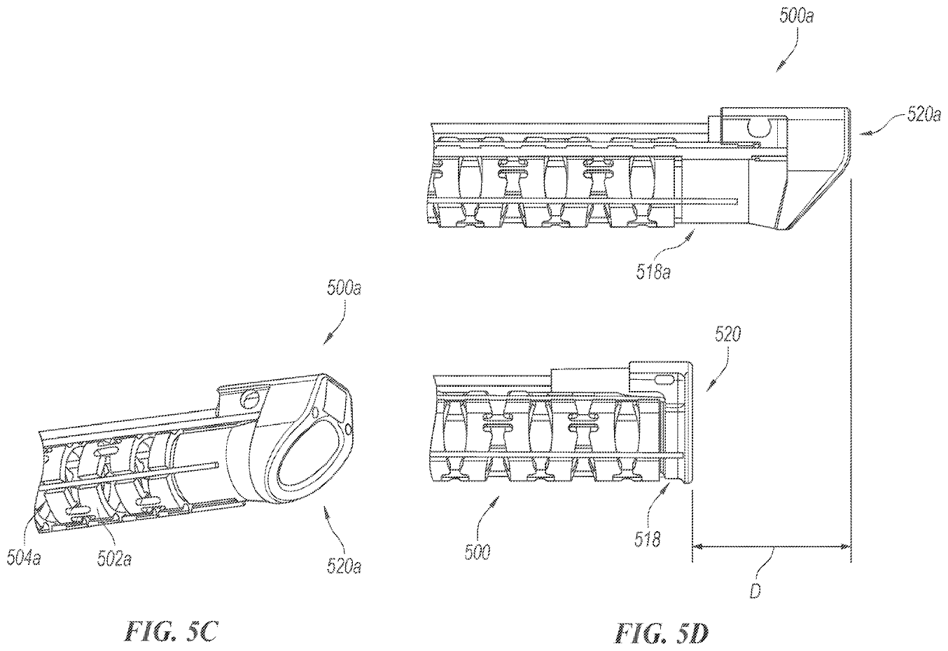

FIGS. 5A and 5B represent an example of a flexible elongate device 500 including components that are provided in a number that cannot be evenly distributed around a perimeter of the flexible elongate device while maintaining a main lumen. FIG. 5A is a perspective view of a distal section of the flexible elongate device 500, while FIG. 5B is a simplified cross-sectional diagram of the distal tip of the flexible elongate device 500. The flexible elongate device 500 can include several features similar to the features of the flexible elongate device 100 described above.

Referring to FIGS. 5A and 5B together, the flexible elongate device 500 can include an axial support structure 502 with a main lumen 504 defined therethrough. The main lumen 504 is provided to allow for delivery of tools during a procedure. In some embodiments, the axial support structure 502 has grooves 506 along its outer surface allowing for components or lumens containing components. For example, a cable bundle 508 for a camera 510 (or camera head) is contained within a secondary lumen 512 located in one of these grooves 506. Similarly, a pair of illumination fibers 514 that provide light to a target site for better visualization by the camera are contained within corresponding secondary lumens 512 located in corresponding grooves 506 along opposing sides of the main lumen 504.

While the secondary lumen 512 is illustrated having a circular cross-sectional shape and the camera (or camera head) 510 and corresponding camera exit aperture (shown within a distal element 520) are illustrated with a square cross-sectional shape, it should be understood that the cross-sectional shapes of the secondary lumen 512 and/or camera 510 may be of any shape including circular, oval, square, rectangular, or any other polygon shape. Generally, the cable bundle 508 and illumination fibers 514 are configured to float within the corresponding lumens such that each component is fixed only at or near the distal end of the flexible elongate device 500. Such an approach allows for minimal impact during bending of the flexible elongate device 500 since these components are carried in lumens having similar profiles as the control elements 516. The camera 510 and distal ends of illumination fibers 514 may terminate at the distal end of the flexible elongate device 500 and be positioned substantially flush to the distal element 520 as illustrated in FIG. 5A. In alternative embodiments, however, the camera 510 and/or distal ends of illumination fibers 514 may extend beyond or terminate just proximal to the distal end of the flexible elongate device 500 or distal element 520. Further, in some embodiments, a diffuser (not shown) may be positioned distally to the distal end of each of the illumination fibers 514 for diffusion of the light as it exits the flexible elongate device 500. In some embodiments, the diffuser may be a mixture of optical adhesive and aluminum oxide. In other embodiments, however, the diffuser may be composed of other suitable materials and/or have a different arrangement.

In the illustrated embodiment, the cable bundle 508 for the camera 510 and illumination fibers 514 can run along their own corresponding lumens down the length of the flexible elongate device 500. In other embodiments, however, the cable bundle 508 and illumination fibers 514 may run within a single lumen down the length of the flexible elongate device 500. Alternatively, the cable bundle 508 for the camera 510 and illumination fibers 514 could be incorporated into a flex cable that runs down the working lumen 504 of the flexible elongate device 500 to a proximal end of the elongate device (not shown). In further embodiments, the flex cable can exit a wall of the elongate flexible device 500 at a distal portion of the device 500 and run along the outside of the elongate flexible device 500 covered by a stretchable/pliant thin latex material/layer. Such an arrangement is expected to eliminate the need to create a specific channel within the flexible elongate device 500 along the spine. In still other embodiments, the cable bundle 508 and/or the illumination fibers 514 may have a different arrangement/configuration along the length of the elongate flexible device 500.

At the proximal end portion (not shown) of the flexible elongate device 500, the cable bundle 508, illumination fibers 514, and control elements 516 may all be operably coupled to a connector (e.g., a vision probe connector) that exits a proximal wall of the elongate flexible device 500. In some embodiments, a proximal housing to which the flexible elongate device 500 is operably coupled can include (a) a duckbill valve positioned along or near a channel through the flexible elongate device 500 to seal liquid from flushing back through the device and (b) one or more T-connectors to allow the device to incorporate both suction and flushing. In lieu of the one or more T-connectors, the proximal housing may alternatively include a touhy valve or, in some instances, a slightly tighter friction fitting between the housing and device may suffice. In additional embodiments, the proximal end portion of the device may also include one or more pockets for receiving portions of the cable bundle 508/illumination fibers 514 to protect these sensitive components from inadvertent exposure to liquids or other undesirable environmental contaminants. In other embodiments, however, the proximal end portion of the device and/or the proximal housing may have a different arrangement/configuration.

Referring back to FIG. 5A and as discussed above, a shape sensor 522 may also extend along the length of the flexible elongate device 500 within a secondary lumen (not shown). The shape sensor 522 may be responsible for tracking the distal section of the flexible elongate device 500.

The control elements 516 for providing actuation control can extend along the length of the flexible elongate device 500 from a distal mount 518 (also referred to as a “control ring”) at the distal tip to corresponding actuators at the proximal end of the flexible elongate device 500. Each actuator may be operable to move the distal section of the flexible elongate device 500 in a single degree of freedom or multiple degrees of freedom. In this example, as best seen in the simplified diagram of FIG. 5B, four control elements 516 are spaced evenly around the circumference of the flexible elongate device 500.

While some components, such as the control elements 516, can be symmetrically distributed around the perimeter of the flexible elongate device 500, other components will be provided in a number that cannot be evenly distributed while maintaining the main lumen 504. For example, incorporating the camera 510, cable bundle 508, and illumination fibers 514 cause the cross section at the distal trip of the flexible elongate device 500 to be non-symmetrical. With an asymmetric design, the moment of inertia will vary when the distal section of the flexible elongate device 500 is articulated along different planes. This is due to the increased stiffness of the flexible elongate device 500 along the side(s) having an imbalanced number of components.

To provide illumination, the pair of illumination fibers 514 may be positioned orthogonally (90 degrees separated) from the camera 510 and camera bundle 508 as illustrated in FIGS. 5A-5B. In some embodiments, not shown, the illumination fibers may be positioned at a farther circumferential location from the camera bundle 508, e.g., providing for larger than 90 degrees of separation between the camera bundle 508 and each of the illumination fibers 514. The distribution of the illumination fibers 514 from the camera bundle 508 may provide a more balanced symmetry of components. In some embodiments, the location of the illumination fibers 508 relative to the camera bundle 508 can be based on the stiffness of each of the illumination fibers 514 relative to the camera bundle 508.

In some embodiments, the illumination fibers 514 provide more effective lighting when positioned at a closer circumferential location to the camera 510. Accordingly, the illumination fibers 514 could be integrated into the cable bundle 508 such that the illumination fibers 514 are positioned adjacent to the camera 510 or the illumination fibers 514 may be positioned within the secondary lumen 512.

FIG. 5C illustrates another example of a flexible elongate device 500a configured in accordance with embodiments of the present technology. In particular, FIG. 5C is a perspective view of a distal region of the flexible elongate device 500a. The flexible elongate device 500a can include several features similar to the features of the flexible elongate devices 100 and 500 described above. For example, the flexible elongate device 500a comprises an axial support structure 502a with a main lumen 504a defined therethrough. The flexible elongate device 500a differs from the devices described previously in that the flexible elongate device 500a comprises a tapered distal tip portion 520a. Without being bound by theory it is expected that, in some procedures, the tapering can help improve access to narrower airways within a patient. The tapered distal portion 520a, however, is an optional feature that may not be present in some embodiments.

FIG. 5D is a simplified side view of the distal tip portion of the flexible elongate device 500a along with a side view of the distal tip portion of the flexible elongate device 500 described previously with reference to FIGS. 5A and 5B. As illustrated, the flexible elongate device 500a having the tapered distal tip portion 520a and corresponding control ring portion 518a are longer in an axial direction (by a distance D) than the flexible elongate device 500. As shown, the distal tip portion of the shorter flexible elongate device 500 comprises a flat or generally flat tip (rather than tapered) with the corresponding control region 518 largely integrated within the distal tip portion of the flexible elongate device 500. Without being bound by theory, it is further expected that, in some procedures, the shortened length of the flexible elongate device 500 with the flat tip region can improve access to narrower airways within some patients.

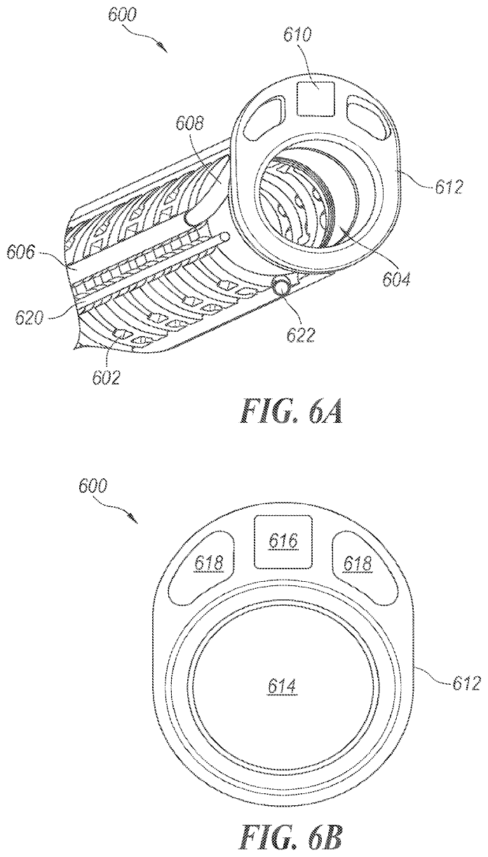

In some embodiments, symmetry can be maintained while optimizing illumination of the target site by directing or routing components of a flexible elongate device near the distal tip to enable more symmetrical bending throughout a proximal section and most of a distal section of the flexible elongate device. FIG. 6A, for example, is a perspective view of a flexible elongate device 600 with illumination fibers 608 that have been routed along the proximal section and most of the distal section of the flexible elongate device 600 at a circumferential location orthogonal to a camera/camera bundle 610 but routed to a circumferential location closer to the camera (or camera head) 610 near the distal tip. In this example, the flexible elongate device 600 includes a pair of illumination fibers 608. However, other embodiments of the flexible elongate device 600 may include more than two illumination fibers 608 or fewer than two illumination fibers 608. For instance, the flexible elongate device 600 may include a single illumination fiber that has been rerouted near the distal tip.

Much like the flexible elongate device 500 described above with reference to FIGS. 5A and 5B, the flexible elongate device 600 shown in FIG. 6A includes an axial support structure 602 with a main lumen defined 604 therethrough. The components of the flexible elongate device 600 are however routed from a first circumferential location at a proximal section to a different circumferential location near the distal tip as described above. Accordingly, a pair of lumens 606 having illumination fibers 608 therein may be placed in grooves along circumferentially opposite sides of the axial support structure 602 and then routed near the distal tip so that the distal end of each illumination fiber 608 is located immediately adjacent to the camera 610.

A distal element 612 having apertures defined therein may be used to help route the illumination fibers 608. As shown in FIG. 6B, the distal element 612 may include an aperture 614 that substantially conforms (e.g., in terms of shape and size) with the main lumen 604, an aperture 616 through which the camera 610 extends, and a pair of apertures 618 through which the illumination fibers 608 extend. While the camera 610 and corresponding camera exit aperture 616 are illustrated with a square cross-sectional shape and the camera bundles, camera bundle lumens, illumination fibers 608, and illumination fiber lumens are shown as circular, it should be understood that all cross-sectional shapes may be of any shape including circular, oval, square, rectangular, or any other polygon shape. While the lumens containing the illumination fibers 608 may be orthogonal to the lumen containing the cable bundle for the camera 610, the apertures 616, 618 for the illumination fibers 608 and camera 610 may be adjacent to one another. Such an approach may cause the cross-sectional shape of the flexible elongate device 600 to be non-symmetrical about a central plane along which the pair of lumens 606 lie. The camera 610 and distal ends of illumination fibers 608 may terminate at the distal end of the flexible elongate device 600 and be positioned substantially flush to the distal element 612 as illustrated in FIG. 6A. In alternative embodiments the camera 610 and/or distal ends of illumination fibers 608 may extend beyond or terminate just proximal to the distal end of the flexible elongate device 600 or distal element 612.

A plurality of control elements 620 may extend along the axial support structure 602. The control elements 620 are spaced circumferentially about the axial support structure 602. In some embodiments the control elements 620 are spaced radially in an even spacing, while in other embodiments the control elements 620 are spaced radially in an uneven spacing. In some embodiments, the flexible elongate device 600 further includes a shape sensor 622 that is located in another groove of the axial support structure 602. The groove containing the shape sensor 622 and the groove containing the cable bundle for the camera 610 may be formed on circumferentially opposite sides of the axial support structure 602. The shape sensor 622 may be positioned at a circumferential location optimizing a balanced symmetry.

Referring back to FIG. 6A, the cable bundle for the camera 610 may extend along the axial support structure 602 in a substantially parallel direction to the control elements 620. Meanwhile, a proximal portion of each illumination fiber 608 may extend along the axial support structure 602 in a substantially parallel direction to the cable bundle for the camera 610 and the control elements 620, while a distal portion of each illumination fiber 608 may extend in a non-parallel direction to the cable bundle for the camera 610 and the control elements 620.

In additional embodiments, the elongate flexible device 600 may comprises a tapered distal tip portion (like the tapered arrangement shown and described above with reference to FIG. 5C) or shortened distal tip portion (like the arrangement shown and described above with reference to FIG. 5D).

B. Preload Controls Scheme for Flexible Elongate Device

Another approach to recovering symmetry involves altering the controls scheme for managing the control elements of the flexible elongate device. One option, for example, involves changing the actuation force applied to each control element so that when all control elements are at a given tension (e.g., minimum tension), the series of control elements will collectively apply a bending moment that counters the increased load. This approach may be referred to as “preloading” because when the flexible elongate device is commanded to enter a limp state, the increased load will be compensated by the tension of some control elements being higher than other control elements.

In determining the amount of actuation force required to effectively preload, a control system can initially determine a correction factor to compensate for the asymmetric condition of the flexible elongate device (e.g., flexible elongate device 500). The control system may determine the correction factor by establishing the tensions needed to maintain a given tip position. As an example, the control system may move the distal tip of the flexible elongate device to a zero position and then measure the tension that must be applied to each control element to maintain the zero position. The term “zero position,” as used here, may be used to refer to any position from which calibration may start. One example of a zero position is the straight position in which the distal tip is experiencing no bending. Said another way, the control system may determine the tension that must be applied to each control element to ensure that the distal tip of the flexible elongate device experiences net zero torque. Note that while in some embodiments, the correction factor could be established or updated at any time, in general, the correction factor is established while the flexible elongate device is located outside of a living body in free space, for example, during a manufacturing or calibration stage.

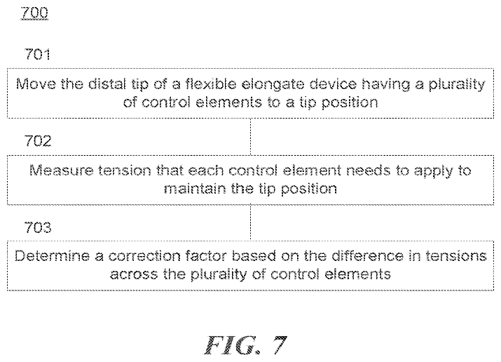

FIG. 7 is a flow diagram of a method 700 for determining the correction factor to compensate for the asymmetric condition of a flexible elongate device having a plurality of control elements in accordance with embodiments of the present technology. The method 700 is illustrated as a set of steps, operations, or processes 701-703, and is described with additional reference to FIG. 5A. By executing the method 700, a control system can characterize the tensions that must be applied to the control elements of the flexible elongate device to compensate for the asymmetric condition. Collectively, the tensions are representative of the correction factor. The method 700 may be executed by a control system during manufacturing (i.e., prior to deployment). Additionally or alternatively, the method 700 may be executed by the control system during runtime prior to a procedure (e.g., as part of a calibration operation).

For the purpose of illustration, assume that a completely symmetric flexible elongate device with a plurality of control elements is to be controlled to minimum tension (T_{MIN}). To maintain this flexible elongate device in the zero position, identical preload tensions can be applied to the plurality of control elements because the flexible elongate device will naturally have net zero torque at its distal tip (and thus a straight neutral position). When a component is added to the flexible elongate device that increases the load in an asymmetric manner, however, the flexible elongate device will no longer have net zero torque at its distal tip. Instead, the flexible elongate device will experience natural bending toward the load and natural resistance to bending away from the load. To counter the load, preload tensions can be applied to the plurality of control elements in accordance with a correction factor. As discussed above, the correction factor will alter the tension symmetry so that when the flexible elongate device is in the zero position, the distal tip will again experience net zero torque. Such correction can be performed independent of the active loading strategy that is employed. Examples of active loading strategies include controlling to minimum tension (T_{MIN}), maximum tension (T_{MAX}), and mid-point tension (T_{MID}). Thus, the “preload tensions” applied to the plurality of control elements to counter the asymmetrical load may be unequal minimum tensions or unequal non-minimum tensions. Further information on controlling to different tensions can be found in International Application No. PCT/US2018/050151, which is hereby incorporated by reference in its entirety.

One aspect to consider is that the correction factor will essentially “consume” some of the total tension budget available to each control element since some control elements will have higher preload tensions than others. The total tension budget can be thought of as the range from the minimum tension that prevents slack to the maximum tension that results in breakage.

Beginning at step 701, a control system can be initially configured without compensation (e.g., without a correction factor or any pre-load) to move the distal tip of the flexible elongate device to a tip position. To accomplish this, the control system may control actuators operably coupled to the control elements such that the tension applied to each control element does not fall below minimum tension (T_{MIN}). Accordingly, each control element may always experience some tension while the flexible elongate device is in use to prevent slack that delays responsiveness. Minimum tension (T_{MIN}) may be set in the programming of the control system.

At step 702 of the method 700, the control system can measure and record the tension that each control element needs to apply to maintain the tip position. For example, to maintain the tip position, the control system may discover that a first amount of tension must be applied to a first control element or subset of the control elements while a second amount of tension must be applied to a second control element or subset of the control elements. Thereafter, at step 703, the control system can determine a correction factor based on the difference in tensions across the plurality of control elements and/or a correlation of the pull wire tensions measured at specific bend angles. At a high level, the correction factor causes unequal tensions to be applied to the plurality of control elements as part of a preload condition designed to compensate for the asymmetric load experienced by the flexible elongate device at the distal tip.

In some embodiments, the control system moves the distal tip of the flexible elongate device to a single tip position, such as the zero position to determine the correction factor. In other embodiments, the control system moves the distal tip of the flexible elongate device to multiple tip positions and to determine a separate correction factor for each tip position. For example, the control system may move the distal tip of the flexible elongate device along one or more commanded planes in incremental movements such as 30-degree increments, 15-degree increments, 10-degree increments, etc. In some embodiments, actuation of the distal tip of the flexible elongate device is in terms of pitch and yaw. In some embodiments, the incremental movements bend the distal tip of the flexible elongate device in positive and negative directions. For example, the distal tip can bend positive 30 degrees in the yaw direction then negative 30 degrees in the yaw direction, and/or positive 30 degrees in the pitch direction then negative 30 degrees in the pitch direction. Thus, the method 700 could be performed multiple times in succession to determine correction factors for multiple tip positions. In such embodiments, the control system can create a coupling matrix (also referred to as a “calibration matrix” or “characterization matrix”) that specifies, for each control element, a minimum level of tension for a plurality of different positions of the distal tip of the flexible elongate device. The characterization matrix may be representative of a data structure that includes the correction factor(s) generated for the flexible elongate device.

In one embodiment, the distal tip position is determined by commanded distal tip position. Additionally or alternatively, the distal tip position is determined by measured distal tip position determined from data generated by a position sensor (e.g. a localization sensor or set of localizations sensors such as EM, or fiber optic sensors which measure shape) that extends along and or couples to the flexible elongate device to determine the position of the distal tip. The position sensor can be used to measure the articulation plane versus the commanded plane as the distal tip of the flexible elongate device is moved. In some embodiments, the characterization matrix is generated by determining the position of the distal tip based on the data generated by the shape sensor.

As discussed above, the preload tensions in the characterization matrix may be generated experimentally by correlating bend of the flexible elongate device with tension in the control elements. In some embodiments, the characterization matrix can include calculated data points in addition to the measured data point by using a linear or sinusoidal fit of bend versus tension to calculate tensions at intermediate bend positions between measured bend positions. For example, tension may be measured and recorded for different bend positions at increments of 30 degrees from negative 90 degrees to positive 90 degrees to create an initial characterization matrix. The characterization matrix can then be updated to include calculated tensions in 15 degree increments within the negative 90 degree to positive 90 degree range by extrapolating tensions using linear or sinusoidal fits of the measured data. In alternative embodiments, the measured tension can be used to generate curves for compensation correction factors representing bends for single degrees of freedom or surfaces representing 2 degrees of freedom measurements, providing tensions as a function of bend angles.

Alternatively or in addition to providing correlation with a static bend position, the characterization matrix can be created to include dynamic effects by applying varying velocities and/or accelerations and then performing a sinusoidal characterization. The velocities and/or accelerations can be commanded velocities/accelerations or can be calculated from data generated by the shape sensor of the flexible elongate device. In one example, an initial characterization matrix or characterization curve/surface can be generated based on static bend measurements as described above. The flexible elongate device can then be commanded to move through specified trajectories at different velocities/accelerations applying a static correction factor based on the initial characterization matrix/curve/surface. A dynamic characterization matrix/surface/curve can then be generated providing a function of flexible elongate device position versus velocity. The dynamic characterization matrix/surface/curve can be used to generate a dynamic correction factor to be applied during operation of the flexible elongate device.

In some embodiments, each characterization matrix could be populated a single time using a representative flexible elongate device and then subsequently applied to similar flexible elongate devices, assuming manufacturing processes are substantially consistent. In some embodiments, the preload tensions are characterized for each flexible elongate device to create personalized characterization matrices, each of which can be made accessible to the corresponding medical instrument system. For example, the personalized characterization matrix created for a given flexible elongate device (e.g., as part of a calibration operation) may be stored in the memory of the medical instrument system of which the given flexible elongate device is a part.

In some embodiments, the preload tensions are calculated based on the stiffness and geometries of the components (e.g., illumination fibers, cable bundles, and shape sensors) included in the flexible elongate device. Accordingly, instead of measuring the tension needed to maintain the distal tip in various tip positions, the characterization matrix may be created using models and/or feedback linearization, where the stiffness of the component(s) causing asymmetry is independently characterized and then the effect of the stiffness is used to calculate the appropriate preload tensions. A feedback-based model may be useful for more complex forms of asymmetry that result in asymmetric behavior that is a function of tip angle. Characterization of the component(s) could be determined experimentally, or characterization of the component(s) could be calculated based on mechanical properties.

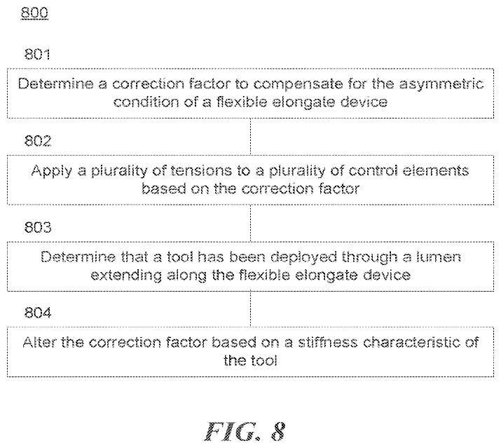

After determining the correction factor for a flexible elongate device, the correction factor can be applied during operation of the flexible elongate device to compensate for an asymmetric condition. FIG. 8 is a flow diagram illustrating a method 800 for operating a flexible elongate device with a plurality of control elements in an asymmetric condition in accordance with embodiments of the present technology. The method 800 is illustrated as a set of steps, operations, or processes 801-804, and is described with additional reference to FIG. 5A.

Beginning at step 801, a control system can initially determine a correction factor to compensate for the asymmetric condition of the flexible elongate device (e.g., flexible elongate device 500). As previously discussed with reference to FIG. 7, the control system may determine the correction factor by discovering the tensions needed to maintain a given tip position.

In some embodiments, the correction factor may be based on or altered by other information describing a condition of the corresponding flexible elongate device. Examples of such information include age, number of uses, number of cleanings, and the like. A new correction factor may be determined or the correction factor determined at step 801 may be altered based on this information. In some embodiments, the new correction factor or alterations are determined empirically by measuring the effect of flexible elongate devices of different ages, number of uses, and number of cleanings. In other embodiments, the alterations are calculated based on the impact that age, uses, and/or cleanings have on the stiffness of the flexible elongate device and its components.

In some embodiments, the control system can determine the type or condition of the flexible elongate device to determine which correction factor to apply or how to alter the correction factor. For example, the type of flexible elongate device and information regarding the flexible elongate device condition may be saved on a memory device coupled to the flexible elongate device. Accordingly, the medical instrument system may be able to automatically detect the flexible elongate device itself and recognize type and condition. For example, the medical instrument system may be able to detect the type of flexible elongate device to be used, the condition (e.g., age, number of uses, number of cleanings, number of sterilizations, etc.), and then establish/derive an appropriate correction factor based on the type and condition of flexible elongate device.

At step 802, the control system can apply a plurality of tensions to the plurality of control elements (e.g., control elements 516) based on the correction factor. These tensions may be referred to as “preload tensions.” As discussed above, the correction factor will provide for maintaining the plurality of tensions at unequal preloads to compensate for asymmetry. Such an approach is expected to cause maximum tension (T_{MAX}) to be achieved more quickly by control elements set at a higher preload tension than those set at a lower preload tension. To counteract this, some control elements (e.g., those arranged in the half plane opposite the load being countered, such as control element 516 of FIG. 5A that is proximate to the shape sensor 522) may be mechanically designed/selected to achieve a higher maximum tension (e.g., have a thicker diameter, be comprised of different materials, etc.).

In some embodiments, the control system is able to monitor whether tools have been deployed through a lumen extending through the flexible elongate device and may alter the correction factor responsive to the determination that an asymmetric tool has been inserted into the main lumen or another lumen. In one embodiment, the correction factor may be altered in real time. In an alternative embodiment, a series of correction factors may be created for different tools. For example, different types of asymmetric tools associated with different purposes can be empirically characterized and then the resulting correction factor can be stored in a datastore. The appropriate correction factor can be obtained by the control system responsive to determining the type of tool that is presently being used. For example, upon determining that the operator has indicated that a given type of asymmetric tool will be deployed through the main lumen of a flexible elongate device, the corresponding control system may access the datastore to obtain the correction factor associated with the given type of asymmetric tool.

Accordingly, at step 803 of the method 800, the control system may determine that a tool has been deployed through the lumen. For example, an operator may be responsible for indicating that a tool has been inserted into the lumen (e.g., by inputting information related to the tool or by scanning a human- or machine-readable code associated with the tool). As another example, the medical instrument system of which the flexible elongate device is a part may be able to automatically detect when the tool has been inserted into the lumen (e.g., with optical sensors, pressure sensors, electromagnetic sensors, and the like). In some embodiments, the system may be able to determine (e.g., based on operator input, sensor readings, or information saved on the tool within a memory device) a type of tool inserted within the lumen. Further information on automatic detection of tools can be found in International Application No. PCT/US2019/030974, which is hereby incorporated by reference herein in its entirety.

In some embodiments, the tool may be inserted within a main lumen extending centrally through the elongate flexible device. The tool may inserted in a manner where it is not held along a central axis of the elongate flexible device or the tool may have an asymmetric construction causing an additional asymmetric load on the elongate flexible device. In another embodiment, an offset lumen (a lumen offset from the central axis of the flexible elongate device) providing an additional asymmetric mechanical load to the flexible elongate device.

Continuing at step 804, if the tool is asymmetric or is inserted within an offset lumen, the control system can alter the correction factor based on a stiffness characteristic of the tool. To establish the stiffness characteristic, the control system may need to obtain, infer, or generate information regarding the tool. For example, the stiffness characteristic may be established based on the type of tool deployed through the lumen. As another example, the stiffness characteristic may be established based on condition of the tool as measured by its age, number of uses, or number of cleanings. Alteration of the correction factor may also be necessary if the tool itself is asymmetric.

To establish what impact, if any, the tool deployed through the lumen will have on the load at the distal tip of the flexible elongate device, the control system may access and/or employ a model able to determine the impact. For example, the model may include one or more algorithms that take information related to the tool (e.g., type, model, age, number of uses, number of cleanings, number of sterilizations, etc.) as input and then produce an estimate of the expected load as output. Additionally or alternatively, the control system may save a datastore, lookup table, or matrix that includes results of past testing of various types of tools under different conditions. To populate the datastore, a series of tests may be performed in which a flexible elongate device performs a calibration operation with tools having different stiffness characteristics contained therein. For example, a first calibration operation may be performed while a first tool of a first age is deployed in the flexible elongate device, a second calibration operation may be performed while a second tool of a second age is deployed in the flexible elongate device, etc. Determinations regarding whether alteration of the correction factor is necessary (and, if so, the amount of alteration that is needed) can be made based on the results contained in the datastore.

The impact of the tool on the load at the distal tip of the flexible elongate device could also be affected by which lumen the tool is inserted within. Accordingly, depending on the type of tool, the system may determine which lumen the tool has been inserted based on the geometric configuration of the flexible elongate device and the intended use of the various lumens. As an example, the system may establish the lumen into which the tool has been inserted based on knowledge regarding the type of tool (e.g., some tools may only be suitable for the main lumen, while other tools may be suitable for an offset lumen). The type of tool may be determined based on operator input, sensor input, or information saved on the tool within a memory device. In an alternative embodiment, sensors (optical sensors, pressure sensors, electromagnetic sensors, and the like) may be used to detect which lumen a tool has been inserted.

Although the steps of the method 800 are discussed in a particular order, the method 800 illustrated in FIG. 8 is not so limited. In other embodiments, the method 800 can be performed in a different order. In these and other embodiments, any of the steps of the method 800 can be performed before, during, and/or after any of the other steps of the method 800. Moreover, a person of ordinary skill in the relevant art will recognize that the method 800 can be altered and still remain within these and other embodiments of the present technology. For example, one or more steps of the method 800 can be omitted and/or repeated in some embodiments.

Another option involves experimentally characterizing the correction factor necessary for the preload condition in real time while the flexible elongate device is in use. For example, the control system may monitor data generated by a shape sensor to determine that the actual position of the distal section of the flexible elongate device matches the intended position where the distal section is supposed to be located. In such embodiments, the control system can alter the preload tensions as necessary such that the actual position matches the intended position. If a fault were to occur due to loss of an actuator, the control system may detect the loss (e.g., by discovering that the position of the distal section has changed without an accompanying command to do so) and then apply an altered correction factor to change the damping on each actuator to balance the load and prevent “snapping back” into the patient anatomy.

### Overview of Medical Instrument Systems

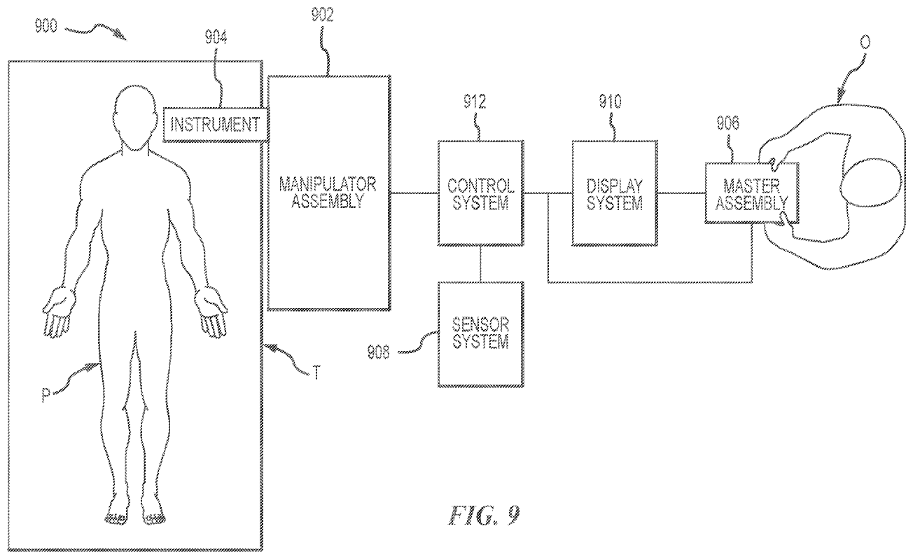

FIG. 9 is a simplified diagram of a teleoperated medical system 900 configured in accordance with various embodiments of the present technology. The medical system 900 may be suitable for use in, for example, surgical, diagnostic, therapeutic, or biopsy procedures. While embodiments may be described herein with respect to such procedures, any reference to medical or surgical instruments or medical or surgical methods is non-limiting. The present technology may be used for animals, human cadavers, animal cadavers, portions of human or animal anatomy, and non-surgical diagnosis, as well as for industrial systems and general robotic, general teleoperations, and robotic medical systems.

As shown in FIG. 9, the medical system 900 generally includes a manipulator assembly 902 for operating a medical instrument 904 in performing various procedures on a patient P. The medical instrument 904 may include, for example, the flexible elongate device 100, 500, and/or 600 of FIGS. 1A, 5A, and 6A respectively. The medical instrument 904 may extend into an interventional site within the body of patient P via an opening in the body of the patient P. The manipulator assembly 902 may be teleoperated, non-teleoperated, or a hybrid teleoperated and non-teleoperated assembly with select degrees of freedom of motion that may be non-motorized and/or non-teleoperated. The manipulator assembly 902 is mounted to or near an operating table T. A master assembly 906 allows an operator 0 to view the interventional site and control the manipulator assembly 902.

The manipulator assembly 902 supports the medical instrument 904 and may include a kinematic structure of one or more non-servo controlled links (e.g., one or more links that may be manually positioned and locked in place, generally referred to as “set-up structure”) and/or one or more servo controlled links (e.g., one or more links that may be controlled in response to commands from the control system 912), and a manipulator. The manipulator assembly 902 may include a plurality of actuators or motors that drive inputs on the medical instrument 904 in response to commands from the control system 912. The actuators may include drive systems that, when coupled to the medical instrument 904, may advance the medical instrument 904 into a naturally or surgically created anatomic orifice. Other drive systems may move the distal end of the medical instrument 904 in multiple degrees of freedom, which may include the three degrees of linear motion and/or three degrees of rotational motion. Additionally, the actuators can be used to actuate an articulable end effector of the medical instrument 904.

The medical system 900 may include a sensor system 908 with one or more sub-systems for receiving information about the manipulator assembly 902 and/or the medical instrument 904. Such sub-systems may include a position/location sensor system (e.g., an electromagnetic (EM) sensor system); a shape sensor system for determining the position, orientation, speed, velocity, pose, and/or shape of a distal end and/or of one or more segments along a flexible body that make up the medical instrument 904; a visualization system for capturing images from the distal end of the medical instrument 904 such as from camera 510 or 610 of FIGS. 1A and 5A respectively; and actuator position sensors such as resolvers, encoders, potentiometers, and the like that describe the rotation and orientation of the motors controlling the medical instrument 904.

The medical system 900 also includes a display system 910 for displaying images or representations of the intervention site and/or the medical instrument 904. The display system 910 and master assembly 906 may be oriented so that the operator 0 can control the medical instrument 904 and the master assembly 906 with the perception of telepresence.

The medical system 900 may also include the control system 912. The control system 912 can include at least one memory and at least one processor for effecting control between the medical instrument 904, master assembly 906, sensor system 908, and display system 910. The control system 912 may also include programmed instructions (e.g., a non-transitory machine-readable medium storing the instructions) to implement some or all of the methods described in accordance with aspects disclosed herein, including instructions for providing information to the display system 910. While the control system 912 is shown as a single block in FIG. 9, the control system 912 may include two or more data processing circuits with one portion of the processing being performed on or adjacent to the manipulator assembly 902, another portion of the processing being performed at the master assembly 906, and/or the like. The processor(s) of the control system 912 may execute instructions corresponding to the processes disclosed herein.

FIG. 10A is a simplified diagram of a medical instrument system 1000 configured in accordance with various embodiments of the present technology. The medical instrument system 1000 includes an elongate flexible device 1002, such as a flexible elongate device 100, 500, and/or 600 of FIGS. 1A, 5A, and 6A respectively, coupled to a drive unit 1004. The elongate flexible device 1002 includes a flexible body 1016 having a proximal end 1017 and a distal end or tip portion 1018. The medical instrument system 1000 further includes a tracking system 1030 for determining the position, orientation, speed, velocity, pose, and/or shape of the distal end 1018 and/or of one or more segments 1024 along the flexible body 1016 using one or more sensors and/or imaging devices as described in further detail below.

The tracking system 1030 may optionally track the distal end 1018 and/or one or more of the segments 1024 using a shape sensor 1022. The shape sensor 1022 may optionally include an optical fiber aligned with the flexible body 1016 (e.g., provided within an interior channel (not shown) or mounted externally). The optical fiber of the shape sensor 1022 forms a fiber optic bend sensor for determining the shape of the flexible body 1016. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. Pat. Nos. 7,781,724, 7,772,541, and 6,389,187, which are all incorporated by reference herein in their entireties. In some embodiments, the tracking system 1030 may optionally and/or additionally track the distal end 1018 using a position sensor system 1020. The position sensor system 1020 may be a component of an EM sensor system with the position sensor system 1020 including one or more conductive coils that may be subjected to an externally generated electromagnetic field. In some embodiments, the position sensor system 1020 may be configured and positioned to measure six degrees of freedom (e.g., three position coordinates X, Y, and Z and three orientation angles indicating pitch, yaw, and roll of a base point) or five degrees of freedom (e.g., three position coordinates X, Y, and Z and two orientation angles indicating pitch and yaw of a base point). Further description of a position sensor system is provided in U.S. Pat. No. 6,380,732, which is incorporated by reference herein in its entirety. In some embodiments, an optical fiber sensor may be used to measure temperature or force. In some embodiments, a temperature sensor, a force sensor, an impedance sensor, or other types of sensors may be included within the flexible body. In various embodiments, one or more position sensors (e.g. fiber shape sensors, EM sensors, and/or the like) may be integrated within the medical instrument 1026 and used to track the position, orientation, speed, velocity, pose, and/or shape of a distal end or portion of medical instrument 1026 using the tracking system 1030.

The flexible body 1016 includes a channel 1021 sized and shaped to receive a medical instrument 1026. FIG. 10B, for example, is a simplified diagram of the flexible body 1016 with the medical instrument 1026 extended according to some embodiments. In some embodiments, the medical instrument 1026 may be used for procedures such as imaging, visualization, surgery, biopsy, ablation, illumination, irrigation, and/or suction. The medical instrument 1026 can be deployed through the channel 1021 of the flexible body 1016 and used at a target location within the anatomy. The medical instrument 1026 may include, for example, image capture probes, biopsy instruments, ablation tools, irritation/aspiration tools, and/or other surgical, diagnostic, or therapeutic tools, including a second flexible instrument (e.g., catheter or flexible elongate device 100 of FIG. 1A, flexible elongate device 500 of FIG. 5A, or flexible elongate device 600 of FIG. 6A) described above. The medical instrument 1026 may be advanced from the opening of channel 1021 to perform the procedure and then be retracted back into the channel 1021 when the procedure is complete. The medical instrument 1026 may be removed from the proximal end 1017 of the flexible body 1016 or from another optional instrument port (not shown) along the flexible body 1016.

The flexible body 1016 may also house cables, linkages, or other steering controls (not shown) that extend between the drive unit 1004 and the distal end 1018 to controllably bend the distal end 1018 as shown, for example, by broken dashed line depictions 1019 of the distal end 1018. In some embodiments, at least four cables are used to provide independent “up-down” steering to control a pitch of the distal end 1018 and “left-right” steering to control a yaw of the distal end 1018. Steerable elongate flexible devices are described in detail in U.S. Pat. No. 9,452,276, which is incorporated by reference herein in its entirety. In various embodiments, medical instrument 1026 (e.g., flexible elongate device 100 of FIG. 1A, flexible elongate device 500 of FIG. 5A, or flexible elongate device 600 of FIG. 6A) may be coupled to drive unit 1004 or a separate second drive unit (not shown) and be controllably or robotically bendable using steering controls.

The information from the tracking system 1030 may be sent to a navigation system 1032 where it is combined with information from the image processing system 1031 and/or the preoperatively obtained models to provide the operator with real-time position information. In some embodiments, the real-time position information may be displayed on the display system 910 of FIG. 9 for use in the control of the medical instrument system 1000. In some embodiments, the control system 912 of FIG. 9 may utilize the position information as feedback for positioning the medical instrument system 1000. Various systems for using fiber optic sensors to register and display a surgical instrument with surgical images are provided in U.S. Pat. No. 8,900,131, which is incorporated by reference herein in its entirety.

In some embodiments, the medical instrument system 1000 may be teleoperated within the medical system 900 of FIG. 9. In some embodiments, the manipulator assembly 902 of FIG. 9 may be replaced by direct operator control. In some embodiments, the direct operator control may include various handles and operator interfaces for hand-held operation of the instrument.

### EXAMPLES

Several aspects of the present technology are set forth in the following examples. Although several aspects of the present technology are set forth in examples directed to systems, computer-readable mediums, and methods, any of these aspects of the present technology can similarly be set forth in examples directed to any of systems, computer-readable mediums, and methods in other embodiments.

1. A non-transitory, computer-readable medium storing instructions thereon that, when executed by one or more processors of a computing system, cause the computing system to perform operations comprising:
receiving a signal to move a distal end portion of a flexible elongate device to a plurality of distal tip positions by actuating a plurality of control elements coupled to the distal end portion of the flexible elongate device, wherein the flexible elongate device is configured to be inserted within an anatomic region of a patient; and
recording a plurality of measured tensions in the plurality of control elements to maintain each of the plurality of the distal tip positions.

2. The non-transitory, computer-readable medium of example 1 wherein the operations further comprise:
storing a correction factor that is based, at least in part, on the plurality of measured tensions; and
receiving a signal to steer the flexible elongate device towards a target by applying the correction factor to a plurality of preload tensions actuating the plurality of control elements, wherein the plurality of preload tensions are maintained at unequal loads.

3. The non-transitory, computer-readable medium of example 2 wherein the plurality of preload tensions are minimum tensions.

4. The non-transitory, computer-readable medium of example 2 wherein the operations further comprise:
determining a type of the flexible elongate device, wherein the correction factor is based at least in part on the type of the flexible elongate device.

5. The non-transitory, computer-readable medium of example 2 wherein the operations further comprise:

determining a condition of the flexible elongate device, wherein the correction factor is based at least in part on the condition of the flexible elongate device.

6. The non-transitory, computer-readable medium of example 5 wherein the condition of the flexible elongate device is at least one of an age of the flexible elongate device, a number of uses of the flexible elongate device, a number of cleanings of the flexible elongate device, or a number of sterilizations of the flexible elongate device.

7. The non-transitory, computer-readable medium of example 2 or example 3 wherein the operations further comprise:
determining a tool is inserted through a lumen of the flexible elongate device;
determining a type of the tool; and
determining the tool affects the asymmetric load condition.

8. The non-transitory, computer-readable medium of example 7 wherein the correction factor is based, at least in part, on the type of the tool.

9. The non-transitory, computer-readable medium of example 8 wherein the operations further comprise:
determining a condition of the tool, wherein the correction factor is based at least in part on the condition of the tool.

10. The non-transitory, computer-readable medium of any one of examples 1-9 wherein the operations further comprise:
creating a calibration matrix specifying each of the plurality of measured tensions correlated with a corresponding distal tip position of the plurality of distal tip positions.

11. The non-transitory, computer-readable medium of any one of examples 1-10 wherein the operations further comprise:

creating a plurality of curves correlating each of the plurality of measured tensions with a corresponding distal tip position of the plurality of distal tip positions.

12. The non-transitory, computer-readable medium of any one of examples 1-11 wherein the plurality of distal tip positions includes a first position and a second position, wherein the second position is in an opposite bending direction from the first position.

13. The non-transitory, computer-readable medium of any one of examples 1-12 wherein the plurality of distal tip positions includes a first position and a second position, wherein the first position is in a yaw direction and the second position is in a pitch direction.

14. The non-transitory, computer-readable medium of any one of examples 1-13 wherein the plurality of distal tip positions includes a zero tip position.

15. The non-transitory, computer-readable medium of any one of examples 1-14 wherein receiving the signal to move the distal end portion of the flexible elongate device includes moving the distal end portion to the plurality of distal tip positions at a plurality of different velocities.

16. The non-transitory, computer-readable medium of example 15 wherein the operations further comprise recording the plurality of different velocities.

17. The non-transitory, computer-readable medium of any one of examples 1-16 wherein the plurality of distal tip positions are each a commanded distal tip position.

18. The non-transitory, computer-readable medium of any one of examples 1-17 wherein the plurality of distal tip positions are determined based on data from a sensor coupled to the distal end portion of the flexible elongate device.

19. A medical instrument system comprising:
a plurality of actuators;
a medical instrument comprising
   a flexible body having a distal end portion,
   a plurality of lumens along the flexible body, wherein the plurality of lumens includes at least one lumen associated with an asymmetric load, and
   a plurality of control elements, each control element coupling the distal end portion to an actuator of the plurality of actuators such that the plurality of actuators is operable to apply tension to the plurality of control elements to move the distal end portion; and
a control system operably connected to the plurality of actuators, the control system being
   configured to execute operations for determining a correction factor comprising
   moving the distal end portion of the medical instrument to a plurality of distal tip positions by actuating the plurality of control elements; and
   recording a plurality of measured tensions in the plurality of control elements to maintain each of the plurality of distal tip positions.

20. The medical instrument system of example 19 wherein the control system is configured to execute further operations comprising steering the medical instrument towards a target by applying the correction factor to a plurality of preload tensions actuating the plurality of control elements, wherein the plurality of preload tensions are maintained at unequal loads.

21. The medical instrument system of example 19 or example 20 wherein the control system is configured to execute further operations for determining the correction factor, the further operations comprising:
creating a calibration matrix specifying each of the plurality of measured tensions correlated with a corresponding distal tip position of the plurality of distal tip positions.

22. The medical instrument system of any one of examples 19-21 wherein the control system is configured to execute further operations for determining the correction factor, the further operations comprising:
creating a plurality of curves specifying each of the plurality of measured tensions correlated with a corresponding distal tip position of the plurality of distal tip positions.

23. The medical instrument system of any one of examples 19-22 wherein each actuator of the plurality of actuators is operable to move the distal end portion of the medical instrument in multiple degrees of freedom of motion.

24. The medical instrument system of any one of examples 19-23 wherein at least one of the plurality of lumens is located along an outer surface of the flexible body and arranged to result in the asymmetric load.

25. The medical instrument system of any one of examples 19-24 wherein the medical instrument further comprises:
a sensor coupled to the flexible body and operable to generate measurements representative of a current configuration of the distal end portion of the medical instrument.

26. The medical instrument of example 25 wherein moving the distal end portion of the medical instrument to the plurality of distal tip positions is based on measurements generated by the sensor.

27. The medical instrument system of example 25 wherein the sensor includes a fiber shape sensor or at least one electromagnetic sensor.

28. The medical instrument system of any one of examples 19-27, further comprising an input device for providing a command for moving the distal end portion of the medical instrument, wherein the plurality of distal tip positions are each a commanded distal tip position.

29. A flexible elongate device, comprising:
a flexible body with an axial support structure having a plurality of grooves arranged thereabout in an asymmetric arrangement;
a distal element having a plurality of apertures,
wherein the axial support structure is proximal to the distal element;
a camera extending within a first aperture of the plurality of apertures and operable coupled to a cable bundle carried within a first groove of the plurality of grooves; and
an illumination fiber positioned within a second groove of the plurality of grooves and extending through a second aperture of the plurality of apertures,
   wherein the second groove is orthogonal to the first groove and the second aperture is adjacent to the first aperture.

30. The flexible elongate device of example 29 wherein the illumination fiber is a first illumination fiber, and wherein the flexible elongate device further comprises:
a second illumination fiber positioned within a third groove of the plurality of grooves and extending through a third aperture of the plurality of apertures,
   wherein the third groove is orthogonal to the first groove and the third aperture is adjacent to the first aperture.

31. The flexible elongate device of example 29 or example 30, further comprising:
a plurality of control elements extending along the flexible body,
   wherein the plurality of control elements is spaced circumferentially about the axial support structure.

32. The flexible elongate device of example 31 wherein the cable bundle extends along the flexible body in a substantially parallel direction to the plurality of control elements.

33. The flexible elongate device of example 31 or example 32 wherein a proximal portion of the first illumination fiber extends along the flexible body in a substantially parallel direction to the cable bundle and the plurality of control elements, and wherein a distal portion of the first illumination fiber extends in a non-parallel direction to the cable bundle and the plurality of control elements.

34. The flexible elongate device of any one of examples 31-33 wherein the plurality of control elements is spaced radially in an uneven spacing.

35. The flexible elongate device of example 34 wherein a first subset of the plurality of control elements are tensioned wires having a first diameter, and wherein a second subset of the plurality of control elements are tensioned wires having a second diameter different than the first diameter.

36. The flexible elongate device of any one of examples 29-35 wherein the flexible body includes a main lumen that extends centrally through the axial support structure and provides a channel for a tool.

37. The flexible elongate device of any one of examples 29-36 wherein a cross-sectional shape of the flexible elongate device is non-symmetrical about a central plane along which the pair of grooves in the axial support structure lie.

38. The flexible elongate device of any one of examples 29-37, further comprising:
a shape sensor located in another groove in the axial support structure,
wherein the groove containing the shape sensor and the groove containing the cable bundle for the camera are formed on circumferentially opposite sides of the axial support structure.

39. A method of determining a correction factor for a flexible elongate device having an asymmetric load condition, the method comprising:
moving a distal end portion of the flexible elongate device to a plurality of distal tip positions by actuating a plurality of control elements coupled to the distal end portion of the flexible elongate device; and
recording a plurality of measured tensions in the plurality of control elements to maintain each of the plurality of the distal tip positions.

40. The method of example 39, further comprising:
storing a correction factor that is based, at least in part, on the plurality of measured tensions; and
steering the flexible elongate device towards a target by applying the correction factor to a plurality of preload tensions actuating the plurality of control elements, wherein the plurality of preload tensions are maintained at unequal loads.

41. The method of example 40 wherein the plurality of preload tensions are minimum tensions.

42. The method of any one of examples 39-41, further comprising:
determining a type of the flexible elongate device, wherein the correction factor is based at least in part on the type of the flexible elongate device.

43. The method of any one of examples 39-41, further comprising:
determining a condition of the flexible elongate device, wherein the correction factor is based at least in part on the condition of the flexible elongate device.

44. The method of example 43 wherein the condition of the flexible elongate device is at least one of an age of the flexible elongate device, a number of uses of the flexible elongate device, a number of cleanings of the flexible elongate device, or a number of sterilizations of the flexible elongate device.

45. The method of any one of examples 39-41, further comprising:
determining a tool is inserted through a lumen of the flexible elongate device;
determining a type of the tool; and
determining the tool affects the asymmetric load condition.

46. The method of example 45 wherein the correction factor is based at least in part on the type of the tool.

47. The method of example 45, further comprising:
determining a condition of the tool, wherein the correction factor is based at least in part on the condition of the tool.

48. The method of any one of examples 39-47, further comprising:
creating a calibration matrix specifying each of the plurality of measured tensions correlated with a corresponding distal tip position of the plurality of distal tip positions.

49. The method of any one of examples 39-48, further comprising:
creating a plurality of curves correlating each of the plurality of measured tensions with a corresponding distal tip position of the plurality of distal tip positions.

50. The method of any one of examples 39-49 wherein the plurality of distal tip positions includes a first position and a second position, wherein the second position is in an opposite bending direction from the first position.

51. The method of any one of examples 39-49 wherein the plurality of distal tip positions includes a first position and a second position, wherein the first position is in a yaw direction and the second position is in a pitch direction.

52. The method of any one of examples 39-51 wherein the plurality of distal tip positions includes a zero tip position.

53. The method of any one of examples 39-52 wherein bending the distal end portion of the flexible elongate device includes moving the distal end portion to the plurality of distal tip positions at a plurality of different velocities.

54. The method of example 53, further comprising recording the plurality of different velocities.

55. The method of any one of examples 39-54 wherein the plurality of distal tip positions are each a commanded distal tip position.

56. The method of any one of examples 39-55 wherein the plurality of distal tip positions are determined based on data from a sensor coupled to the distal end portion of the flexible elongate device.

### CONCLUSION

The systems and methods described herein can be provided in the form of tangible and non-transitory machine-readable medium or media (such as a hard disk drive, hardware memory, optical medium, semiconductor medium, magnetic medium, etc.) having instructions recorded thereon for execution by a processor or computer. The set of instructions can include various commands that instruct the computer or processor to perform specific operations such as the methods and processes of the various embodiments described here. The set of instructions can be in the form of a software program or application. Programmed instructions may be implemented as a number of separate programs or subroutines, or they may be integrated into a number of other aspects of the systems described herein. The computer storage media can include volatile and non-volatile media, and removable and non-removable media, for storage of information such as computer-readable instructions, data structures, program modules or other data. The computer storage media can include, but are not limited to, RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. In one embodiment, the control system supports wireless communication protocols such as Bluetooth, IrDA, HomeRF, IEEE 802.11, DECT, and Wireless Telemetry. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art. The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. Furthermore, the various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Similarly, the phrase “and/or” as in “A and/or B” refers to A alone, B alone, and both A and B. Additionally, the terms “comprising,” “including,” “having” and “with” are used throughout to mean including at least the recited feature(s) such that any greater number of the same feature and/or additional types of other features are not precluded.

Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

From the foregoing, it will also be appreciated that various modifications can be made without deviating from the technology. For example, various components of the technology can be further divided into subcomponents, or various components and functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

1. A non-transitory, computer-readable medium storing instructions thereon that, when executed by one or more processors of a computing system, cause the computing system to perform operations comprising:
receiving a signal to move a distal end portion of a flexible elongate device to a plurality of distal tip positions by actuating a plurality of control elements coupled to the distal end portion of the flexible elongate device, wherein the flexible elongate device is configured to be inserted within an anatomic region of a patient; and
recording a plurality of measured tensions in the plurality of control elements to maintain each of the plurality of the distal tip positions.

2. The non-transitory, computer-readable medium of claim 1 wherein the operations further comprise:
storing a correction factor that is based, at least in part, on the plurality of measured tensions; and
receiving a signal to steer the flexible elongate device towards a target by applying the correction factor to a plurality of preload tensions actuating the plurality of control elements, wherein the plurality of preload tensions are maintained at unequal loads.

3. The non-transitory, computer-readable medium of claim 2 wherein the plurality of preload tensions are minimum tensions.

4. The non-transitory, computer-readable medium of claim 2 wherein the operations further comprise:
determining a type of the flexible elongate device, wherein the correction factor is based at least in part on the type of the flexible elongate device.

5. The non-transitory, computer-readable medium of claim 2 wherein the operations further comprise:
determining a condition of the flexible elongate device, wherein the correction factor is based at least in part on the condition of the flexible elongate device.

6. The non-transitory, computer-readable medium of claim 5 wherein the condition of the flexible elongate device is at least one of an age of the flexible elongate device, a number of uses of the flexible elongate device, a number of cleanings of the flexible elongate device, or a number of sterilizations of the flexible elongate device.

7. The non-transitory, computer-readable medium of claim 2 wherein the operations further comprise:
determining a tool is inserted through a lumen of the flexible elongate device;
determining a type of the tool; and
determining the tool affects the asymmetric load condition.

8. The non-transitory, computer-readable medium of claim 7 wherein the correction factor is based, at least in part, on the type of the tool.

9. The non-transitory, computer-readable medium of claim 8 wherein the operations further comprise:
determining a condition of the tool, wherein the correction factor is based at least in part on the condition of the tool.

10. The non-transitory, computer-readable medium of claim 1 wherein the operations further comprise:
creating a calibration matrix specifying each of the plurality of measured tensions correlated with a corresponding distal tip position of the plurality of distal tip positions.

11. The non-transitory, computer-readable medium of claim 1 wherein the operations further comprise:
creating a plurality of curves correlating each of the plurality of measured tensions with a corresponding distal tip position of the plurality of distal tip positions.

12. The non-transitory, computer-readable medium of claim 1 wherein the plurality of distal tip positions includes a first position and a second position, wherein the second position is in an opposite bending direction from the first position.

13. The non-transitory, computer-readable medium of claim 1 wherein the plurality of distal tip positions includes a first position and a second position, wherein the first position is in a yaw direction and the second position is in a pitch direction.

14. The non-transitory, computer-readable medium of claim 1 wherein the plurality of distal tip positions includes a zero tip position.

15. The non-transitory, computer-readable medium of claim 1 wherein receiving the signal to move the distal end portion of the flexible elongate device includes moving the distal end portion to the plurality of distal tip positions at a plurality of different velocities.

16. The non-transitory, computer-readable medium of claim 15 wherein the operations further comprise recording the plurality of different velocities.

17. The non-transitory, computer-readable medium of claim 1 wherein the plurality of distal tip positions are each a commanded distal tip position.

18. The non-transitory, computer-readable medium of claim 1 wherein the plurality of distal tip positions are determined based on data from a sensor coupled to the distal end portion of the flexible elongate device.

19. A medical instrument system comprising:
a plurality of actuators;
a medical instrument comprising:
a flexible body having a distal end portion,
a plurality of lumens along the flexible body, wherein the plurality of lumens includes at least one lumen associated with an asymmetric load, and
a plurality of control elements, each control element coupling the distal end portion to an actuator of the plurality of actuators such that the plurality of actuators is operable to apply tension to the plurality of control elements to move the distal end portion; and
a control system operably connected to the plurality of actuators, the control system being configured to execute operations for determining a correction factor comprising
moving the distal end portion of the medical instrument to a plurality of distal tip positions by actuating the plurality of control elements; and
recording a plurality of measured tensions in the plurality of control elements to maintain each of the plurality of distal tip positions.

20. The medical instrument system of claim 19 wherein the control system is configured to execute further operations comprising steering the medical instrument towards a target by applying the correction factor to a plurality of preload tensions actuating the plurality of control elements, wherein the plurality of preload tensions are maintained at unequal loads.

21-38. (canceled)
